(19) United States
(12) Patent Application Publication
(10) Pub. No.: US 2020/0286056 A1
(43) Pub. Date: Sep. 10, 2020

(54) FRONT END USER INTERFACE PROCESSING FOR DYNAMICALLY ORIGINATING AND ADJUSTING HEALTHCARE DATA

(71) Applicant: iVinci Partners, LLC, Boise, ID (US)

(72) Inventors: Vincent Martino, Eagle, ID (US); Kent F. Ivanoff, Boise, ID (US)

(21) Appl. No.: 16/808,601

(22) Filed: Mar. 4, 2020

Related U.S. Application Data
(60) Provisional application No. 62/813,549, filed on Mar. 4, 2019.

(51) Int. Cl. G06Q 20/14; G06N 20/00; G06Q 20/10; G16H 40/00

(52) U.S. Cl. CPC G06Q 20/14; G16H 40/00; G06Q 20/102; G06N 20/00

(57) ABSTRACT

Systems and methods of data processing to establish and manage aspects of data transactions for payments, financing, and estimates in connection with healthcare service transactions are disclosed. In an example, healthcare information system data, estimate data, and patient information data may be obtained and processed or accessed for use in a front-end workflow for identifying estimated balance and payment conditions of a scheduled healthcare visit (e.g., before any services have been provided) A trained data processing model or other algorithm may be used to establish a payment arrangement for the estimated balance prior to billing for the healthcare visit, and as applicable, establish a payment schedule modification, refund, or rebilling action based on detecting a deviation between an estimated balance and an actual balance. Various user interface features and workflows are also disclosed to enable accompanying data-driven processes and functionality.

### PRIORITY CLAIM

This application claims the benefit of priority to U.S. Provisional Application Ser. No. 62/813,549, filed Mar. 4, 2019, which is incorporated herein by reference in its entirety.

### TECHNICAL FIELD

The present application discloses various techniques and system configurations to access, identify, acquire, and update data pertaining to healthcare transactions. In specific examples, such techniques and configurations provide user interface functionality relating to the generation and management of specific forms of data values relevant to payment or financing of healthcare transactions.

### BACKGROUND

Computing systems have greatly improved the capability and sophistication of entities to track and manage the collection of payments for healthcare transactions (e.g., doctor visits, medical procedures, drug administration, etc.). However, many forms of healthcare billing systems are fragmented and incomplete and lack functionality to provide a positive customer payment experience as a balance or payment is being managed. Many technical limitations are encountered with this data because current healthcare billing systems are primarily designed to track claims and optimize payments by third-party payers (e.g., insurers) only after a healthcare billing event has taken place. As a result, presently available healthcare billing systems present limited portrayals of data and information, especially when attempting to access or estimate balances of multiple transactions among individual patient accounts.

Many types of healthcare billing systems also are not equipped to elegantly manage many aspects of patient (or guarantor) account balances. Patients are often confused by statements generated by billing systems, especially since many billing systems do not present a guarantor with a single electronic statement that includes all charges incurred for multiple disparate visits and expenses during a single period. This confusion also extends to payment arrangements and estimates that are attempted to be established before the healthcare procedure or activity has occurred. Due to the many sources of payment and myriad potential adjustments to bills (e.g., insurance payments, deductible and co-pays, provider discounts) and the wide variation in medical provider costs and charges, estimates of balances due are imprecise, or are not attempted, for many types of healthcare services.

For providers that do offer estimates, many are hesitant to offer payment financing alternatives because there is no efficient way to deal with the material deviations that nearly always occur between an estimate and the actual balance due, after insurance adjudication. As a result, providers often do not offer payment and financing alternatives even though they realize such payment flexibility is necessary. If such alternatives are made available, providers are forced to manage cumbersome manual processes to make adjustments for balance differences; otherwise, if such adjustments are not implemented by the provider, the consumer must contact the provider themselves to manually “fix” the payment arrangement.

For these and other reasons, many medical providers do not collect pre-payment or offer flexible financing options for patients to accurately plan repayment before healthcare services are provided. Although various service providers have developed systems to offer overall estimates of healthcare charges, prior to the healthcare services, such estimates are often focused on the results of insurance coverage (e.g., to collect an insurance deductible or co-pay) and can only provide a rough estimate of the total amount that a patient or guarantor will have to pay for the services if insurance coverage is applied. Additionally, while healthcare payment plans and financing may be offered and set up by a medical provider prior to healthcare services, a large degree of human judgment and review is needed in order to establish financing terms, identify eligibility for financing or discounts, and reconcile payments made on an estimated payment balance with an actual payment balance. As a result, many healthcare providers are unable to offer useful payment arrangements to patients or guarantors or collect accurate pre-payment before medical services occur.

### BRIEF DESCRIPTION OF THE DRAWINGS

FIG. 1 is a block diagram depicting data transactions conducted among a data processing system and user interfaces, according to an embodiment.

FIG. 2 is a block diagram depicting data transactions and communications conducted among a data processing system, processing functionality, and user interfaces, according to an embodiment.

FIG. 3 is a flowchart of an example method of obtaining and managing data for healthcare visit estimate processing, according to an embodiment.

FIGS. 4A and 4B are respective illustrations of an example administrative user interface for accessing front-end payment information for a patient account, according to an embodiment.

FIGS. 4C to 4G are respective illustrations of example administrative user interface features for implementing front-end payment management for a patient account, according to an embodiment.

FIG. 5 is a flowchart of an example method of generating and managing user input data for healthcare visit estimate processing, according to an embodiment.

FIGS. 6A to 6F are respective illustrations of example patient user interface features for implementing front-end payment management for a patient account, according to an embodiment.

FIG. 7 is a flowchart of an example method of reconciling and managing data for dynamic payment modification, according to an embodiment.

FIG. 8 is an illustration of example patient user interface features for implementing payment modification for front-end payment management for a patient account, according to an embodiment.

FIG. 9 is a flowchart of an example method of originating and adjusting front-end healthcare payment data representations, according to an embodiment.

FIG. 10 is a schematic of data sources and data records operating in a data processing system, according to an embodiment.

FIG. 11 is a schematic of operational and functional components used among devices and systems for implementing functionality of originating and adjusting front-end healthcare payment data representations, according to an embodiment.

FIG. 12 is a block diagram illustrating an example of a machine upon which one or more embodiments may be implemented, according to an embodiment.

### DETAILED DESCRIPTION

Healthcare providers and their patients may encounter payment issues when medical procedures and services are performed that are likely to result in a large patient balance, such as in scenarios where insurance coverage is limited and the patient is likely to owe at least some predetermined amount (e.g., child birth, scheduled surgical procedure, etc.), in scenarios where services are performed unexpectedly (e.g., due to complications or the need for additional procedures), or if procedures are elective in nature (e.g., cosmetic procedures, bariatric procedures, etc.). Given the wide variation in types and costs of medical services and procedures, insurance coverages, and repayment conditions, and because most medical procedures are personal to a patient, a vast variety of payment and billing scenarios may be encountered in healthcare settings.

The present disclosure provides embodiments of computer-implemented systems and methods to address these issues, through the transformation and processing of healthcare transaction data, using specialized graphical user interfaces, payment data processing systems, and related healthcare information systems. Embodiments of the disclosed systems and methods may, for example, utilize customized user interfaces, application programming interfaces (APIs), algorithms and models, and on-demand scoring to enable appropriate front-end payment and financing coordination prior to the occurrence of healthcare services. Such systems and methods may be operated through the use of: patient-facing interfaces (e.g., for use by a guarantor who has financial responsibility for one or more patients); medical provider or financial provider interfaces (e.g., for use by a customer service agent who assists patient customers, or a call center representative who reviews patient accounts); healthcare data reconciliation systems; and other forms of healthcare, financial, or information-related systems.

In a first example, various aspects of data processing are provided through the use of a real-time algorithm that evaluates data inputs collected from a patient or guarantor, healthcare provider, and third party data sources. This real-time algorithm is used to identify and generate payment rules and characteristics in a variety of healthcare workflows. Workflows include those that are used to collect an advance payment, to establish a financing plan, to offer full or partial financial assistance (e.g., as part of charity care), or to establish combinations of payment and finance scheduling. The algorithm may be operated as part of machine-intelligence driven models or processing functionality that offers: the generation of estimates and personalized offers in advance of a scheduled medical visit; coordination and status information for ongoing balances and financing arrangements based on changes to the scheduled medical visits or patient information; updates or status notifications being provided to the patient as the medical visit or set of medical visits occur; changes or modifications being offered and implemented in financing arrangements and scheduled payments; identification of which of the consumer's health savings, banking, or payment accounts and associated payment methods to draw from and in which order, on an automated basis, and like coordination among medical and financial data aspects.

In a second example, various aspects of data processing are provided through the use of a real-time algorithm (or multiple algorithms) that evaluates data that tracks states of financing, and dynamically generates and identifies (and in some examples, implements) new terms for a financing schedule, payment plan, or other financial arrangement. For instance, if a financing balance deviates from the estimate during or after the medical service is provided (e.g., because the balance is smaller than originally estimated within the financing plan), the loan terms within the boundaries of the financing plan may be adjusted. This form of an auto-correction to the payment terms may be automatically adjusted and automatically re-presented to the consumer (e.g., as a notification if the balance has decreased, or for acceptance of revised terms if the balance has increased), or accompanied by other aspects of notifications, prompts, and user inputs to maintain user control.

In other examples, the implementation of algorithms, real-time processing functions, patient information tracking, and healthcare information compilation provides a number of data-driven workflows for healthcare data management functionality. This functionality may enable a medical provider or agent to implement financing and collection strategies including: accurate estimates and payments operations before the medical visit occurs (e.g., as part of scheduling, registration, or pre-authorization workflows); targeted follow-ups during or after the medical visit (e.g., to identify scenarios where the type, quantity, or amount of services or goods rendered in the visit varies from the estimate or prediction); and notifications and information updates of financing and payment status that are coordinated to actual charges.

The presently disclosed embodiments provide a variety of improvements to payment capabilities and payment management operations for a service provider (e.g., a healthcare provider, payment processor, financial institution, etc.), while also introducing an improved billing experience for guarantors and patients to identify and coordinate payment for healthcare transaction charges. However, such improvements are not limited to business or financial aspects, as a variety of technical improvements are experienced through the use of improved data processing operations, coordinated communications, controlled workflows, and information input and outputs.

Further, the presently disclosed embodiments provide a number of technical benefits which enhance the operation of healthcare-specific configured computing systems. These include, the introduction of new user interface workflows which provide efficiency over manual human data entry and payment collection processes; advanced processing algorithms to collect, match, reconcile, and adjust payment data fields and to automatically adjust payment amounts when a balance changes; real-time data processing, which provides accurate information to reduce manual user transactions, unnecessary payment or refund transactions; and dynamic user interfaces which offer new types of controls and information displays on a data-driven basis.

The term “provider” as used herein refers broadly to an entity providing a product or service. A provider may be a single individual (e.g., a physician) providing a benefit or may be an entire organization (e.g., a healthcare organization with multiple subsidiaries, facilities, locations, departments). Thus, a provider is not limited to a single individual or facility, but may refer to a single individual or entity, a group of individuals or entities, multiple locations, subsidiaries, departments (e.g., laboratory, pathology, anesthesiology, etc.), affiliates, and the like.

The term “guarantor” as used herein refers to a person (or entity) that has or will assume primary or at least shared financial responsibility for payment for an amount owed, such as for a product or services rendered (e.g., healthcare services rendered to a patient) at a visit or in connection with the receipt of such product or service. A guarantor makes or gives a promise, assurance, or pledge relating to financial payment performance on a financial liability, either on behalf of himself/herself or on behalf of another. The guarantor, in some instances, may also be the beneficiary of the product or service (e.g., a patient in the case of healthcare services). Another example of a guarantor may be a parent or legal guardian of a minor. Although in some instances laws or regulations may dictate qualifications for a guarantor (e.g., age requirements, mental capacity), the term as used herein is not bound to these restrictions. Further, many references to a “patient” are used interchangeable with references to a “guarantor”.

The term “visit” as used herein refers to an encounter or an instance of receipt of services (or a product). For example, some embodiments of the disclosure are described with reference to healthcare services rendered to a patient, and a visit can be considered a physical visit to a healthcare provider and receipt of healthcare services (or products) rendered during that physical visit. However, the disclosed embodiments are not limited to medical forms of healthcare services. Other examples of healthcare visits relate to dental services, vision services, mental health services, and the like. As can be appreciated, a visit or encounter may not necessarily involve physical movement of the beneficiary to another location, and a visit can include services or products that come to the beneficiary or that are provided from a remote location (e.g., telemedicine) or that are otherwise performed for or directed to the benefit of the beneficiary.

The term “front-end” in the context of a “front-end payment” and “front-end workflows” as used herein primarily refers to the performance of payment actions and workflows that occur before, or concurrent with, a medical visit or other healthcare service (or, in some examples, post-service but before insurance adjudication) where the amount due is not fully known. However, in some settings, such payment actions and workflows may also occur before or concurrent with billing actions (e.g., insurance billing) or other reimbursement workflows.

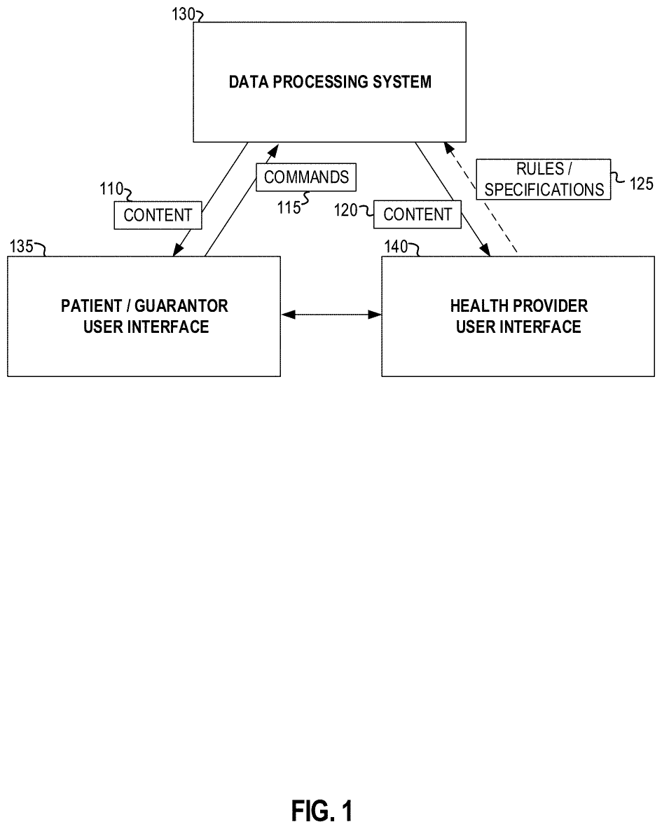

FIG. 1 is a block diagram depicting data transactions conducted among a data processing system 130 and user interfaces 135, 140. As shown, the data processing system 130 exchanges communications with a patient/guarantor user interface 135 and a health provider user interface 140. The patient/guarantor user interface 135 may also exchange communications with a health provider user interface 140 (or, in some examples, the patient/guarantor user interface 135 may be hosted by, or linked to, the health provider user interface 140, as shown with the dashed line).

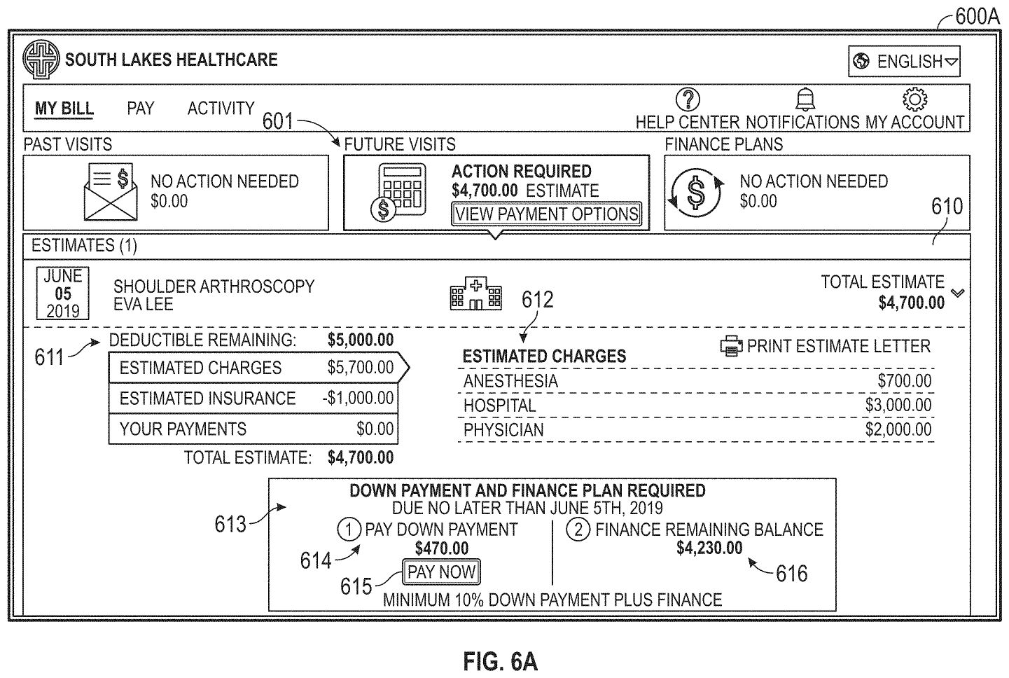

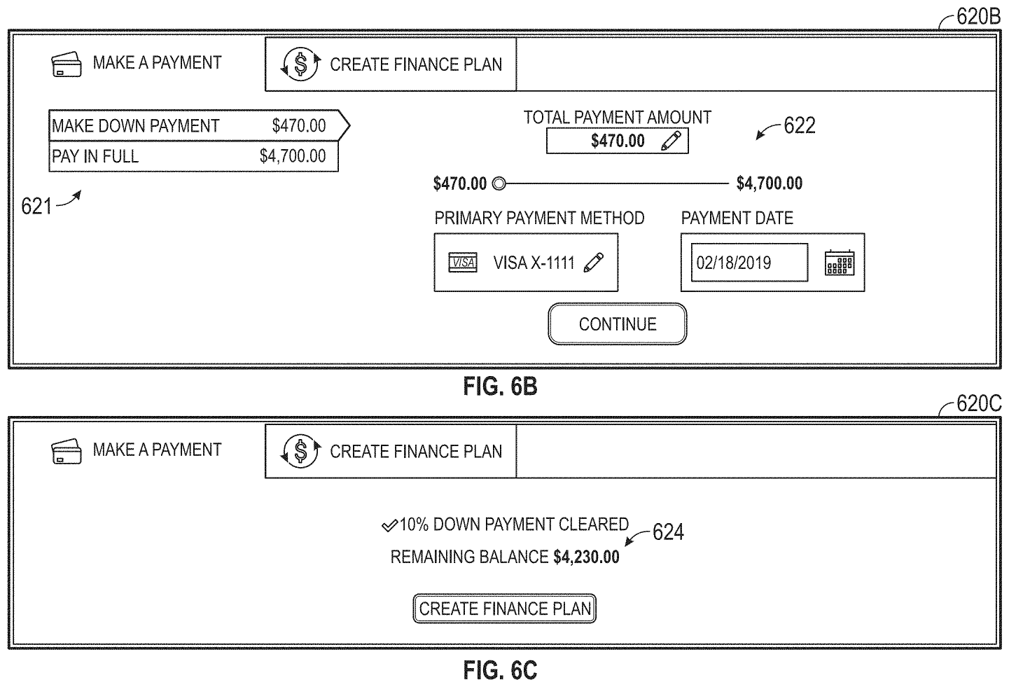

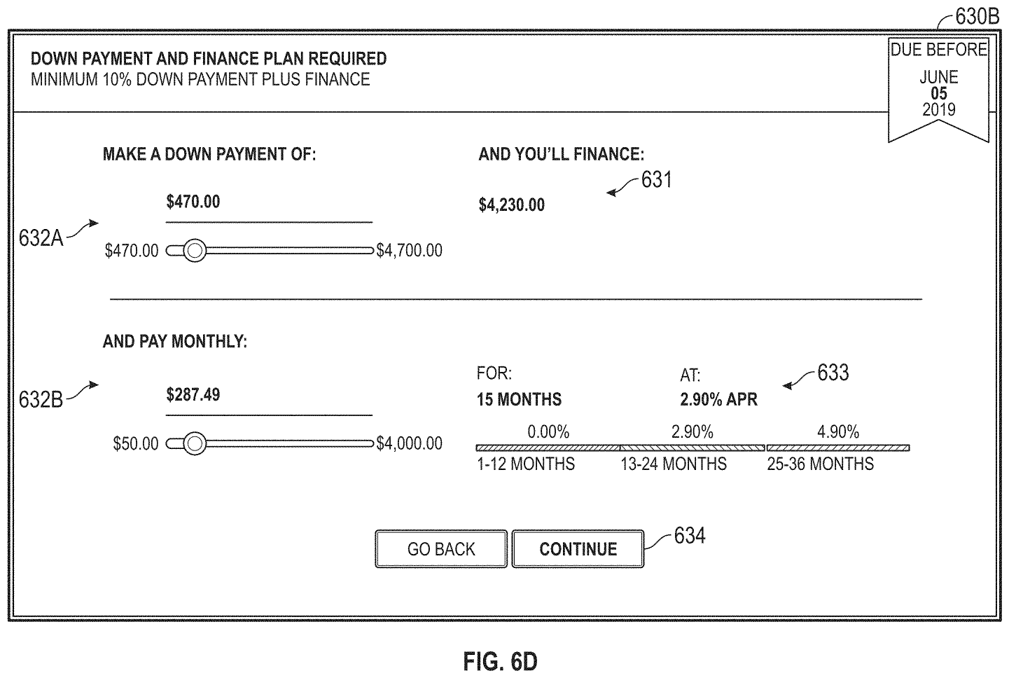

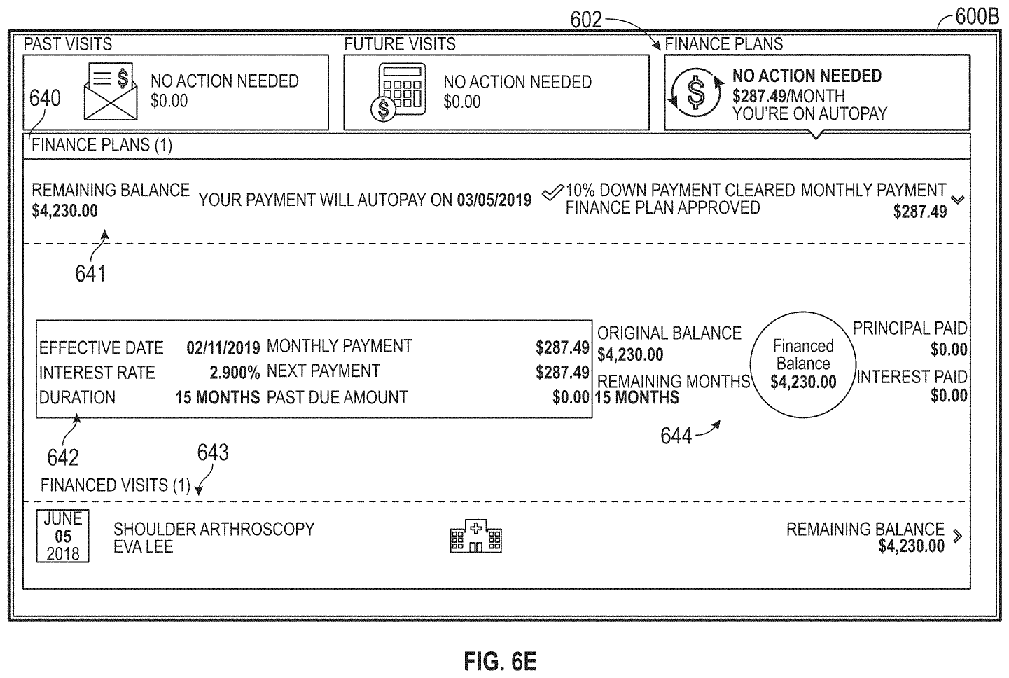

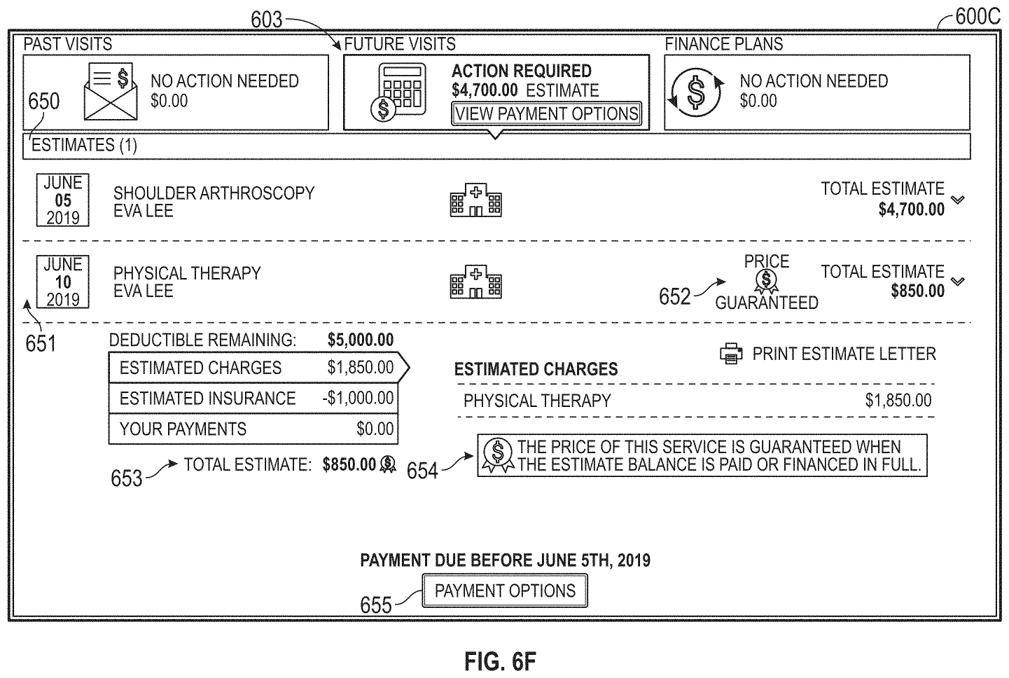

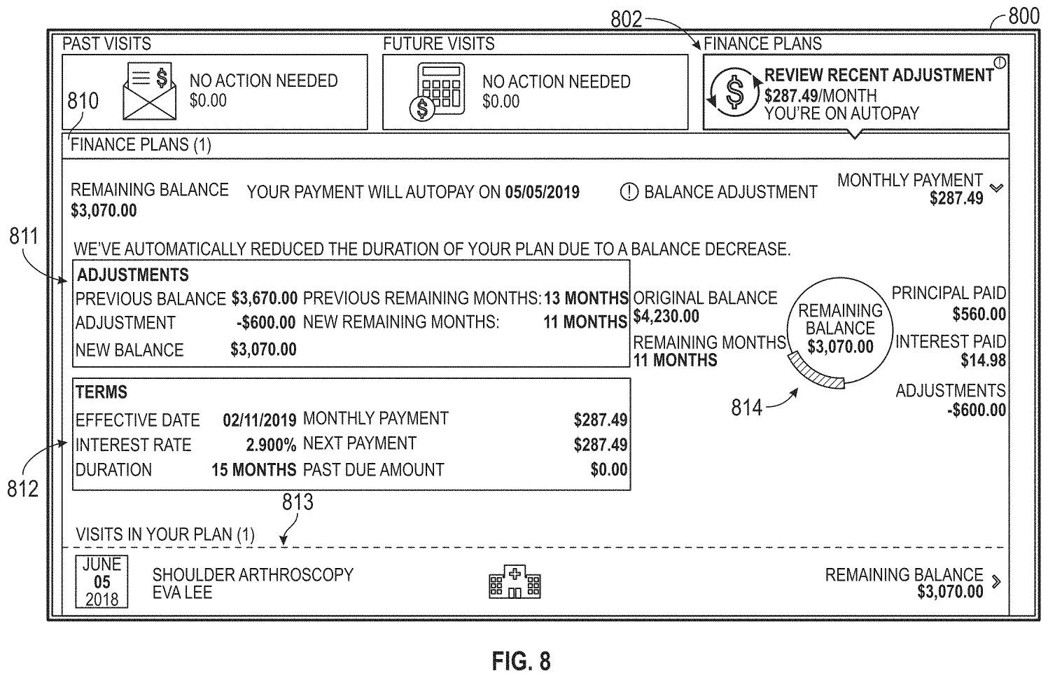

The patient/guarantor user interface 135 is used to output content 110 received from the data processing system 130 (e.g., content related to amounts, conditions, or types of payment or financing). The patient/guarantor user interface 135 is used to input commands 115 to be communicated to the data processing system 130 (e.g., commands to establish, modify, or accept payment or financing operations). Examples of the types of content 110 that are provided within the patient/guarantor user interface 135 are illustrated within the user interface outputs in FIGS. 6A to 6F and FIG. 8. Examples of the types of commands 115 that are communicated to the data processing system 130 include commands triggered from user interface inputs in FIGS. 6A to 6F and FIG. 8.

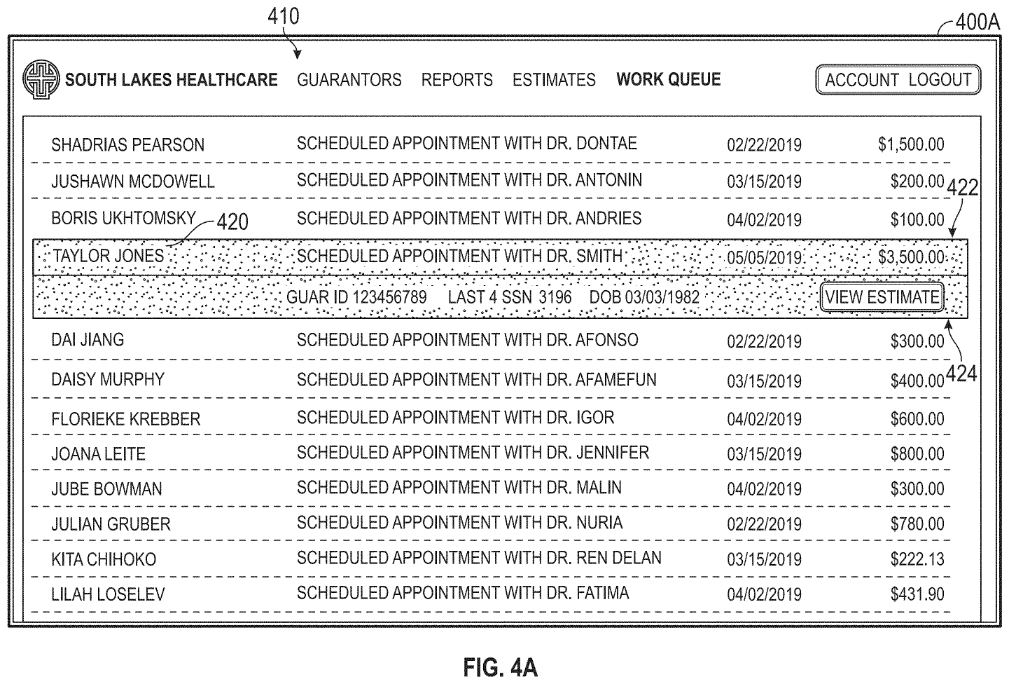

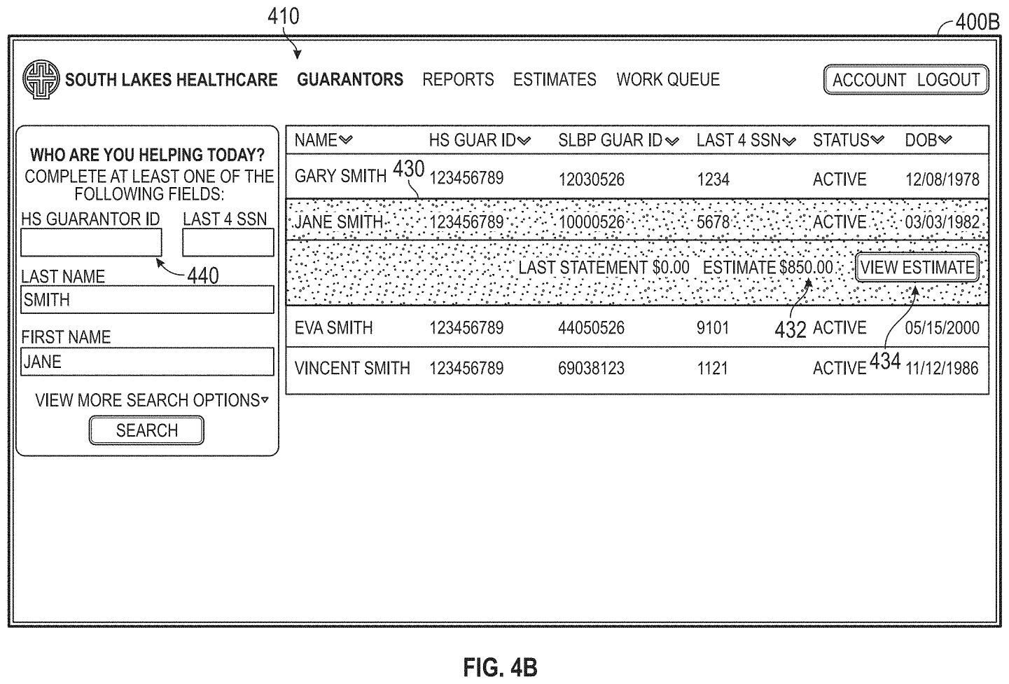

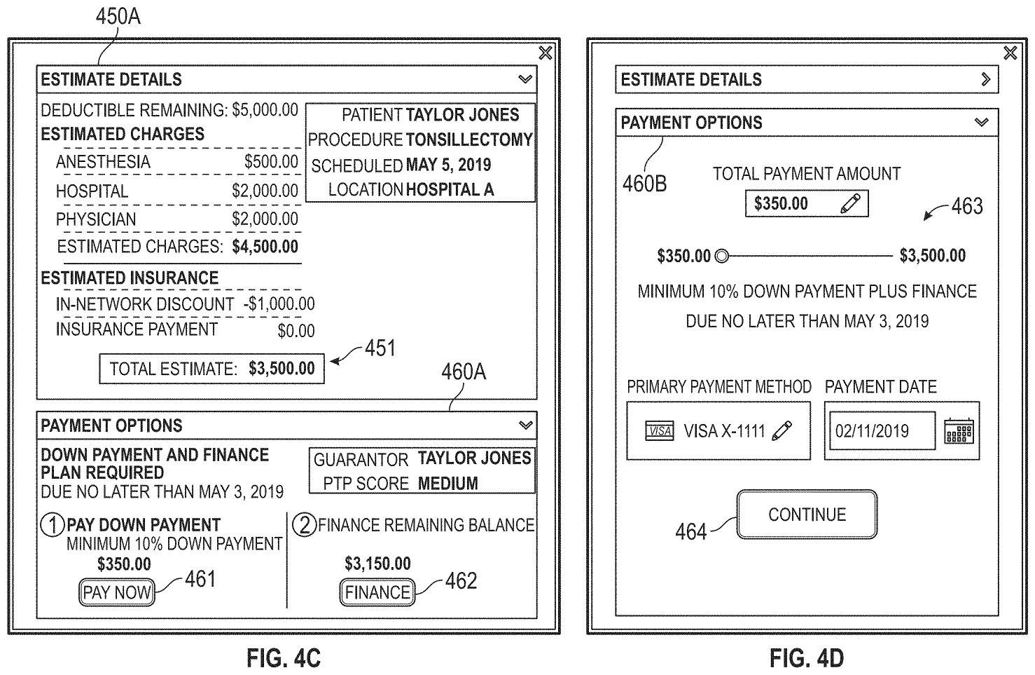

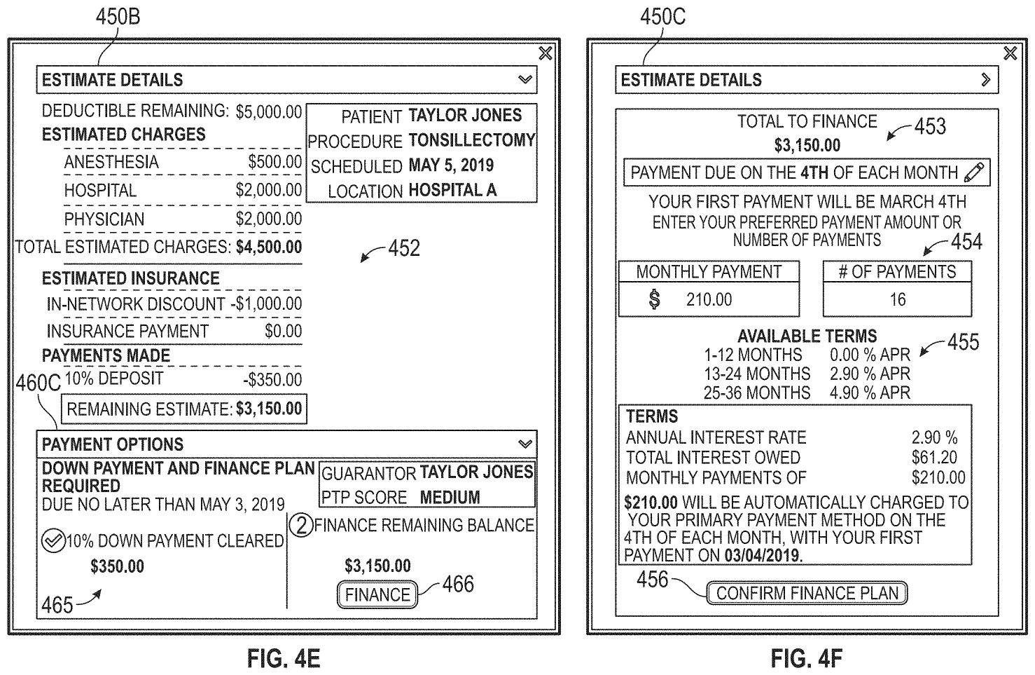

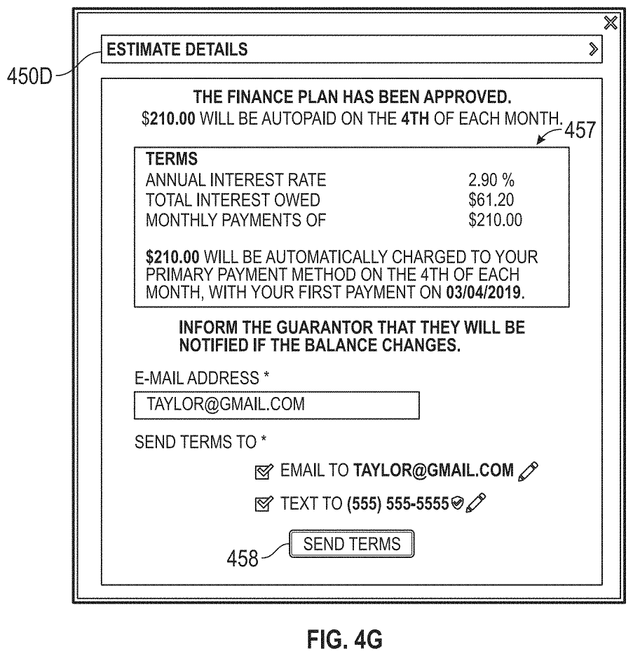

The health provider user interface 140 is likewise used to present content 120 received from the data processing system 130 (e.g., content related to amounts, conditions, or types of payment or financing, for the patient/guarantor, other patients/guarantors, a health provider, the like). The health provider user interface 140 is used to input rules and specifications 125 to communicate to the data processing system 130 (e.g., commands to establish, modify, or accept payment or financing operations). Examples of the types of content that are provided within the administrative user interface, and controls to trigger commands within the administrative user interface, are illustrated within the user interface of FIGS. 4A to 4G.

In some examples, the patient/guarantor user interface 135 is operated exclusively by the patient, guarantor, or a person on behalf of the patient or guarantor, such as where the patient/guarantor user interface 135 provides a limited information display and controls. In other examples, features of the health provider user interface 140 and the patient/guarantor user interface 135 are integrated into a single user interface, or managed by an administrative user interface (not shown in FIG. 1) which may be operated by a user on behalf of the medical provider, a payment provider, or the like.

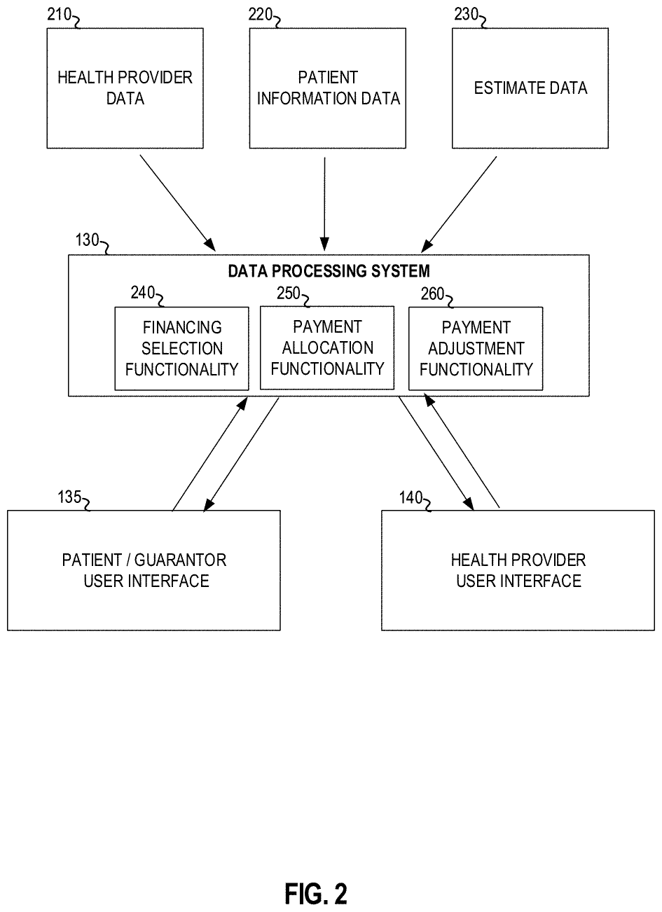

FIG. 2 is a block diagram depicting data transactions and communications conducted among the data processing system 130, and user interfaces 135, 140, with use of processing functionality. The processing functionality enables various data-driven actions to occur for aspects of pre-visit (front-end) financing and payment, and associated billing actions and data transactions.

In an example, the processing functionality of the data processing system 130 includes financing selection functionality 240, payment allocation functionality 250, and payment adjustment functionality 260. For instance, the financing selection functionality 240 may utilize dynamic data operations to select, present, classify, identify, and implement relevant financing terms and conditions (e.g., conditions defined by a medical provider) as part of financing for a healthcare visit or set of visits. The payment allocation functionality 250 may utilize dynamic data operations to select, present, classify, identify, and implement a payment event or schedule, to achieve payment from the patient or guarantor for the healthcare visit or set of visits. The payment adjustment functionality 260 may utilize dynamic data operations to correct, change, modify payment schedules, financing plans, or specifications for the healthcare visit or set of visits, on behalf of the patient or guarantor, the healthcare provider, a payment or billing service provider, or another entity.

The data processing system 130 performs the data-driven operations based on a variety of data sources, which may include, but are not limited to: a health provider data source 210, such as an electronic medical records system which indicates scheduled or historical visits, or a medical record billing system, which indicates historical charges for the patient or relevant visit charges; a patient information data source 220, which indicates information for the patient, such as credit worthiness, demographic information, payment information, or values relevant to a propensity to pay or other scoring metric; and an estimate data source 230, which indicates values relevant to calculating an estimate of charges in advance of a particular visit, healthcare transaction, course of treatment, or the like. The data sources 210, 220, 230 may be managed by the healthcare provider or by separate parties (e.g., by third party financial service companies).

In addition to or in place of the data sources depicted in FIG. 2, the data processing system 130 may be operably or communicatively coupled with a billing data source, a healthcare information data source, a payer (e.g., insurance) data source, a credit data source, a demographics/consumer data source, a payment processing system, or the like. For instance, a billing data source and/or healthcare data source may include information from a billing system (or a plurality of billing systems of one or more client healthcare providers). The healthcare data source may be a health information system (or a plurality of health information systems of one or more client healthcare providers). Any of these data sources may be accessed via a network, including with the use of respective application programming interfaces (APIs) and corresponding API calls and functions.

For instance, billing data sources may include one or more hospital billing systems, clinic billing systems, practitioner billing systems (e.g., physician billing systems), modules within these or similar medical record and billing systems (such as legacy systems that may integrate data from the acute and ambulatory environments), and/or the like. The healthcare data sources may include, but are not limited to: electronic health record systems, Personal Health Record (PHR) systems, Personal Health Information Technology (PHIT) systems, Electronic Health Record (EHR) systems, Health Information Exchange (HIE) systems, and/or the like. The credit data sources may include credit reporting agencies, such as Equifax, Experian, TransUnion, and/or the like. The demographics/consumer data sources may include demographic and/or consumer profiling data sources configured to provide information including, but not limited to: census information (e.g., date of birth, head of household, marital status, ethnicity, age, education level, and the like), household information (e.g., phone number, number of dependents, length of residence, residence status, residence size, residence value, and the like) economic information (e.g., income level, purchasing power, and the like), mortgage information (e.g., mortgage amount, rate, term, and the like), neighborhood information, socio-economic indicators, and so on. In some examples, an extraction engine is provided by or operable with the data processing system 130 to transform, convert, and/or translate information acquired from the data sources 210, 220, 230.

Additional front-end processing functions that are provided, or integrated, with the data processing system 130 may include, but are not limited to: providing business intelligence information to the client healthcare provider, assessing propensity-to-pay scoring information, brokering charges and/or balances to transfer a corresponding accounts receivable asset to a new asset holder, enabling offers of configurable financing and payment terms to beneficiaries and/or guarantors, linking accounts of guarantors across billing systems, and linking accounts of guarantors to an account of a manager guarantor. Among other features, propensity-to-pay information may incorporate information regarding the transaction history of the account guarantor with the healthcare system, condition of the patient, credit reporting information, demographics, and/or the like. Further aspects of such front-end (and back-end) processing functions are described in U.S. patent application Ser. No. 14/590,457 to Ivanoff et al., which is incorporated by reference herein in its entirety.

In an example, the financing selection functionality 240 may utilize aspects of propensity-to-pay scoring in determining available financing and payment options for a particular patient or guarantor. Such scoring may include using a client provider's historical transaction data to help assess the likelihood of future guarantor payments based on the historical payment behaviors of the guarantor being scored or a like guarantor (e.g., having similar payment characteristics and/or attributes). However, this scoring information may be obtained from a third party, or produced with a predictive machine learning classification model. Additionally, propensity to pay and presumptive charity information and characteristics may be obtained and defined by a healthcare provider and/or calculated using behavioral segmentation models and algorithms. Additional aspects of evaluation, criteria, and rules may be applied as part of the financing selection functionality, such as to identify that, based on propensity-to-pay scoring and/or other financial information (e.g., balance history, credit reporting information, demographics, and/or the like), that the guarantor is unlikely to pay for a future visit unless financing terms are established.

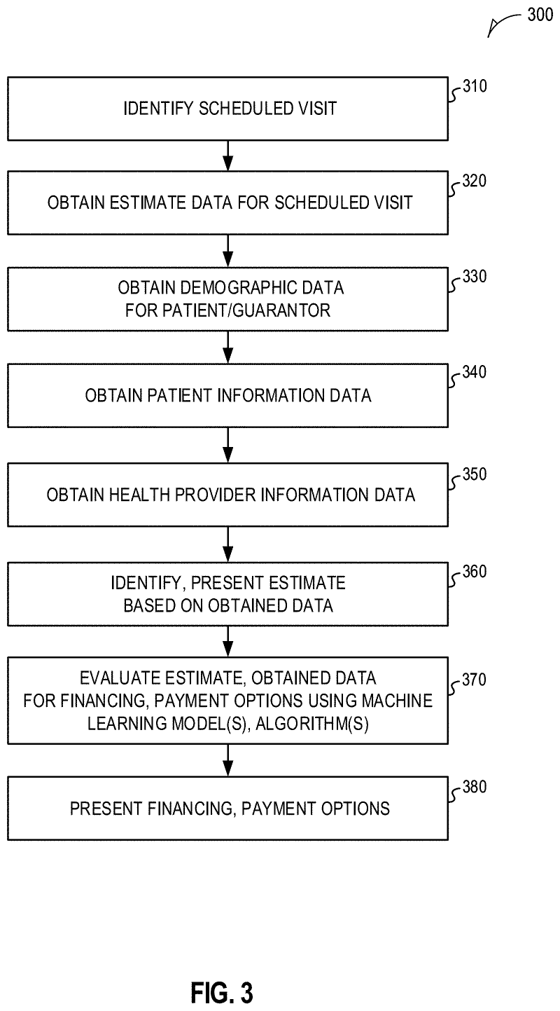

FIG. 3 is a flowchart 300 of an example method of obtaining and managing data for healthcare visit estimate processing, such as for front-end financing and payment management. These operations may be performed in connection with a user interface offering functionality indicated above for patient/guarantor user interface 135 or health provider user interface 140, or as part of the features provided in the user interface illustrations provided in FIGS. 4A to 4G and 6A to 6F, discussed below.

The flowchart 300 begins with operations to identify a scheduled visit or set of visits (operation 310). In an example scenario, a visit is scheduled by the provider and patient, but the healthcare service has not yet been provided (and, billing for the service has not yet occurred). The data processing system connects to a scheduling system to retrieve and identify data that indicates the scheduled visit.

The flowchart 300 continues to obtain estimate data of costs and repayment scenarios for the scheduled visit (operation 320) (e.g., the patient's insurance plan status information, and the status of the patient insurance plan deductible), and to obtain credit, demographic, or other personal data for the patient/guarantor (operation 330). This estimate data may be retrieved from a third party, or, the data processing system may produce the estimate data based on provider information (such as health system policies, reimbursement rates or rules, etc.). The demographic data may be directly obtained from the patient/guarantor or from a third party service, and may include information such as address, a Social Security Number, phone number, and the like, or may include demographic data derived from such fields. In various examples, many of the data retrieval operations 320-350 may occur in parallel; in other examples, some of the data retrieval operations 320-350 may be dependent on one another.

The flowchart 300 continues to obtain patient information data (operation 340) and health provider information data (operation 350). This may include information maintained by a healthcare provider such as existing patient balances, payment history, the healthcare provider's charity policies, repayment thresholds, and other financial data values.

The flowchart 300 continues to identify and present an estimate of the charges for a visit or set of visits, based on the obtained data (operation 360). In an example, this estimate is presented through a user interface which includes medical information related to the visit (e.g., via a healthcare provider website portal). In other examples, the information is provided in a user interface that exclusively provides financial values. As will be understood, the estimate may be provided through a real-time ability to dynamically underwrite a consumer and generate a financing decision and estimate based on myriad factors (score, financial need, balance, type of treatment, etc.), to identify whether a down payment is needed prior to service, and what payment and financing alternatives are available after the down payment for the residual amount due is processed.

The flowchart 300 continues to evaluate the estimate and obtained data, for financing and payment options applicable to the particular patient/guarantor (operation 370). Such options may be provided in the form of a personalized offer, which is generated using a real-time processing algorithm. Such an algorithm may be provided by a machine learning model and data processing algorithms which consider a propensity to pay score or other financial characteristics to develop a personalized, dynamic payment plan offer for the particular patient/guarantor. This algorithm can ensure the offer falls within the bounds of the healthcare provider's policy related to repayment, charity care, discounts, and the like. The algorithm and offer functionality may enable automatic funding of the balance from any balance sheet or creditor, including the healthcare provider themselves, all on a front-end basis before the visit or payment adjudication occurs. As an example, this algorithm can dynamically determine whether a proposed offer falls outside the provider's financing policy, and if the case, identify one or more alternative financing sources and use decisioning logic to cascade the balance to whichever creditor is ultimately willing to make financing available. In still further examples, the algorithm and offer functionality may also involve use of an auction for a financing asset, where creditors bid for the debt and the consumer can decide the best alternative to meet their needs.

The flowchart 300 then concludes by a presentation of financing and payment options (operation 380). For example, an offer may be presented to the patient/guarantor through a device agnostic UI, either directly to the patient or via an agent of the healthcare provider. The offer may also be presented in other communication mediums (e.g., emails, text messages, websites, etc.).

The presented options or offers are presented with certain “boundaries” and clear rules or explanations to the patient, to accommodate the fact that the balance will likely change. In some scenarios, the offer may include a required or optional down payment from 0-100%, a required or optional finance plan up to the number of months the provider desires, a required or optional series of up to recurring payments (such as a number of payments that do not necessitate establishing a finance plan according to legal terms). The finance plan offer may include interest for some scenarios, or discounts for certain payments in some examples. The finance plan or recurring payment start date can be either immediately (pre-service) or be delayed until post service or until the visit is adjudicated (through the payer). Some variations to the options or offer may be user-controlled or selectable.

FIGS. 4A and 4B are respective illustrations of an example administrative user interface for accessing front-end payment information for a patient account. FIG. 4A provides an example selection interface 400A to enable selection of a particular guarantor account from among a list of plurality of guarantors; FIG. 4B provides an example selection interface 400B to enable selection of a particular guarantor account based on a search criteria. In both examples, the interfaces 400A, 400B each include user interface controls 410 for navigation by an administrative user (e.g., customer call center or service representative) to perform administrative functions, and it will be understood that many other types of information outputs and controls may be provided in accompanying user interface screens.

The user interface 400A provides the ability for a selection 420 of a particular guarantor from a list of guarantors, with a summarized display of personal information (e.g., identifiers, date of birth), date of service, and estimated payment amount 422. The summarized display of personal information is also provided with a selection control 424 (e.g., button), which may transition to aspects of the interface screens of FIGS. 4C to 4G.

The user interface 400B provides an ability to search or identify information based on patient or guarantor criteria, such as a search facility 440 which enables a patient or set of patients to be identified based on name, identifier, or the like. The results of the search facility 440 may be provided in a search list, which allows user interaction with one or more patients or guarantors. The user interface 400B also illustrates a selection 430 of a particular guarantor, which then provides a summarized view of personal information and billing information, including an estimated payment amount 432 and a selection control 434 (e.g., button) that allows an estimate to be viewed in detail.

FIGS. 4C to 4G are respective illustrations of example administrative user interface features for implementing front-end payment management for a patient account. For instance, the activation of the selection control 424 or 434 may provide use of a workflow among the respective user interfaces features.

FIG. 4C depicts an estimate detail interface 450A and payment option interface 460A which respectively provide a detailed breakdown of estimated charges for a future visit 451, and available payment options to establish a payment and financing for the future visit. Among other information, the estimate detail interface 450A provides a total estimate used as part of a front-end payment and financing plan.

FIG. 4D depicts another payment option interface 460B which is produced from the selection of a down payment option (e.g., from the selection of control 461). This payment option interface 460B may provide user interactive features to allow the customization of a payment, within a defined payment range (e.g., considering a minimum or maximum range). The payment option interface 460B may also include inputs to receive or select a payment method and payment date. Additionally, the payment option interface 460B is also depicted as including a payment selection control 464 (e.g., button) to schedule the payment.

FIG. 4E depicts another example of an estimate detail interface 450B, which specifies payment terms for a financing. The payment terms may be provided in an itemized list 452. Suggested or recommended payment options 465 and a user interface control 466 (e.g., button) to select or activate financing of a remaining balance may be provided in a payment option interface 460C.

FIG. 4F depicts another example of the estimate detail interface 450C, which is used to receive user input of payment term values for establishing a payment financing. For instance, a specified payment amount and payment schedule 453 may be defined or modified (e.g., by an administrative user). A payment input 454 may suggest or receive a user input for a preferred payment amount or number of payments. The interface 450C may also include an output of available financing terms 455, and details or information on such terms or the proposed financing plan. The confirmation of the terms for the financing plan may be established with the selection of an input control 456 (e.g., button).

FIG. 4G concludes with a further example of an estimate detail interface 450D, which provides a summarized view of financing information 457. This interface may include an input to communicate the financing terms to the user, through activation of a control 458 (e.g., button) and accompanying selection of a delivery media (e.g., to activate an email or text message). This interface 450D may also include instructions or information, such as to enable a call center representative to follow a predefined script to explain the finance plan terms (e.g., a legally or contractually required statement).

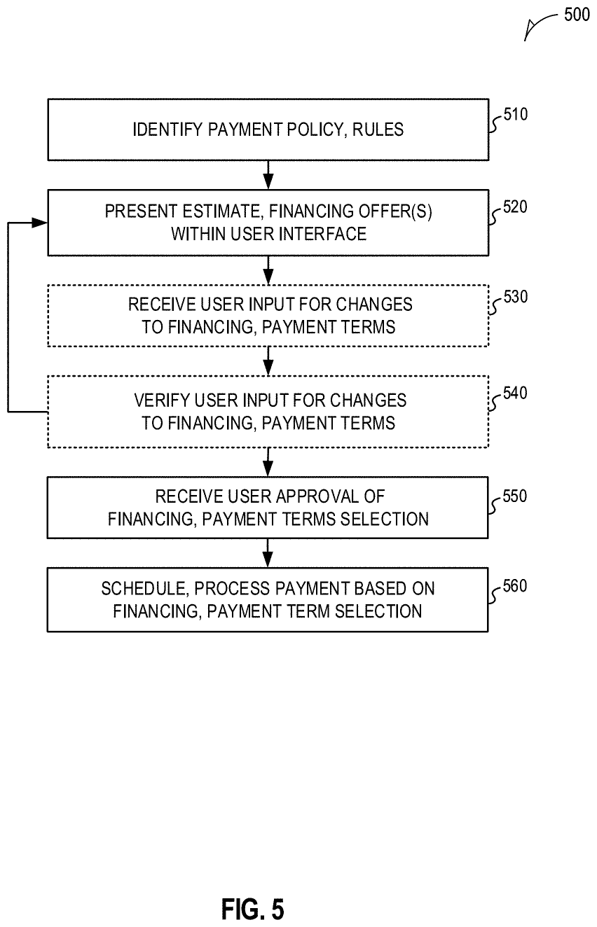

FIG. 5 is a flowchart 500 of an example method of generating and managing user input data for healthcare visit estimate processing, such as for front-end financing and payment selection. The operations of the flowchart 500 may follow or integrate with the operations provided by flowchart 300, discussed above. The operations of the flowchart 500 may occur in connection with workflows triggered through use of a user interface, such as a user interface providing functionality indicated in the illustrations of FIGS. 4A to 4G or 6A to 6F.

The flowchart 500 begins with operations to identify (and, in some examples, store or access) a payment policy and rules associated with financing or repayment scenarios (operation 510). As noted above, a payment policy and rules may be provided based on information associated with the healthcare provider. These inputs may be processed in real-time, using machine learning models and algorithms, to establish a propensity to pay score for the patient and develop a personalized, dynamic payment plan offer for specific visit or visits. The algorithm will ensure that the offer falls within the bounds of the healthcare provider's policy.

The flowchart 500 continues with the presentation of estimate and financing offers, within a user interface (operation 520), such as the interface examples discussed above. For instance, one or more offers may be presented to the patient through a device-agnostic user interface (e.g., a mobile device-compatible user interface), either directly to the patient or via an agent of the provider. As noted above, the offer may be provided with certain boundaries and rules to the patient, to accommodate the fact that the balance will likely change. For instance, the offer may include a required or optional down payment from 0-100%, a required or optional finance plan up to the number of months the provider desires, a required or optional series of recurring payments (e.g., up to four payments, not necessitating a finance plan). Based on characteristics of the user or provider, or applicable regulations or conditions, a finance plan offer may include interest. In further examples, various types of machine learning analysis may be performed to modify the offer being provided or presented to the patient. Such analysis may modify the offer based on historical accuracy of the estimate versus actual (eventual) cost.

The flowchart 500 includes optional operations to receive user input for changes to financing or payment terms (operation 530), and verify user input of changes or values for financing or payment terms (operation 540). For instance, if changes are made, verification is performed to ensure that the terms of the patient's acceptance are compliant with rules and policies. Changes, modifications, or rejection of the terms may cause a new presentation or update of available options (operation 520) (e.g., if the user-entered terms are out of an acceptable range) and the receipt of additional user input for changes and verification of the changes (repeating operations 530, 540).

The flowchart 500 continues with the receipt of user approval for financing and payment terms (operation 550). In connection with user approval, the terms of the financing and payment are tracked and associated with the patient and patient visit. If the patient does not provide immediate approval for the terms, follow up reminders may be provided to the patient to review or modify the financing and payment offer.

The flowchart 500 concludes with the scheduling and processing of a payment (such as a down payment, or payment schedule) based on the financing and payment term selection (operation 560). In various examples, the finance plan or recurring payment start date can be either immediately (pre-service payment), be delayed until post-service, or be delayed until the visit is adjudicated (e.g., reconciled with insurance coverage).

FIGS. 6A to 6F are respective illustrations of example patient user interface features for implementing front-end payment management for a patient account. First, FIG. 6A depicts a user interface 600A which provides a number of accessible billing functions for a guarantor, with a future visit illustration 601 being provided for a particular scheduled medical procedure.

Within the future visit illustration 601, estimated visit charges and financing is output in an estimated visit interface 610. One specific visit is depicted in the estimated visit interface 610, showing the estimate of a specific visit to perform a medical procedure. This visit interface includes an estimated payment and financing overview 611, estimated billing overview 612, and payment and financing offer control 613. Within the payment and financing offer control 613, detail is provided to indicate a required down payment 614, a remaining financing balance 616, and a payment input control 615 (e.g., button) to select a particular payment.

The user interface illustrations provided in FIGS. 6B to 6F illustrate results of a payment workflow to allow a guarantor or patient to directly interact with different aspects of payment and financing, in a similar manner as provided for an administrative user in FIGS. 4C to 4F. FIGS. 6B and 6C provide respective payment interfaces 620B, 620C, used to receive a payment. The user interface 620B provides a mechanism to output payment options 621, and receive a user specified payment 622 (e.g., customizable between a minimum and maximum amount); the user interface 620C provides a mechanism to output and indicate a remaining balance 624.

The user interface illustration provided in FIG. 6D provides a finance plan interface 630B that specifies the terms of a finance plan (in a similar manner as FIG. 4F), with a specified payment amount and payment schedule 631, payment inputs 632A, 632B to suggest, receive, or adjust a preferred payment amount or number of payments (e.g., for a down payment and monthly payment), and an output of selected financing terms 633. The finance plan interface 630B also includes a user-selectable control 634 (e.g., button) allowing the finance plan to be implemented from user input.

FIG. 6E provides a detailed view of user interface 600B for accessing financing plan functionality 602, as part of a display of financing plans interface 640. The financing plan interface 640 presents payment details 641 for an accepted financing plan, terms 642 of the financing plan, a list of visits 643 applicable to the financing plan, and balance information 644 applicable to the financing plan.

FIG. 6F provides a detailed view of an interface 600C for accessing estimate charges functionality 603, as part of a display of an estimate interface 650. The estimate interface 650 presents financing or payment plan details 651 for an accepted financing plan established for multiple estimates, including an indication 652 of price-guaranteed estimate values for a particular estimate. Additionally, the estimate interface 650 presents estimate details 653 for the particular estimate, such as based on estimated charges, estimated insurance coverage, estimated payments, and the like. Further, the estimate interface 650 presents estimated charge details 654 for the particular charges, allowing the user to see the price of individual procedures (including, if applicable, accompanying facility charges, drug charges, equipment charges, etc.) estimated to occur at individual visits.

In various examples, the values generated or output as part of the future visit illustration 601, financing plan functionality 602, estimate charges functionality 603, may be produced with the use of machine learning analysis. For example, analysis of data inputs into a machine learning model may help identify a guaranteed price for a scheduled procedure (e.g., for the guarantee value depicted in FIG. 6F) based on the historical accuracy of the estimate. In further examples, these and similar machine learning analysis may be extended to analyze generated estimates, to be able to set a price point that ensures profitability within a given statistically derived tolerance band for a provider (e.g., a hospital or health system). This may occur by optimizing on both price and expected value considering coverage by payer and collection rate of patient.

Finally, in further examples, other sources of payment may be considered for the guaranteed or generated estimates discussed above. The guaranteed or generated estimates may consider payments expected or scheduled to be received from other third-party payment sources, such as insurance companies, government or non-profit organizations, third party guarantors, medical cost reimbursement or rebate programs, and the like.

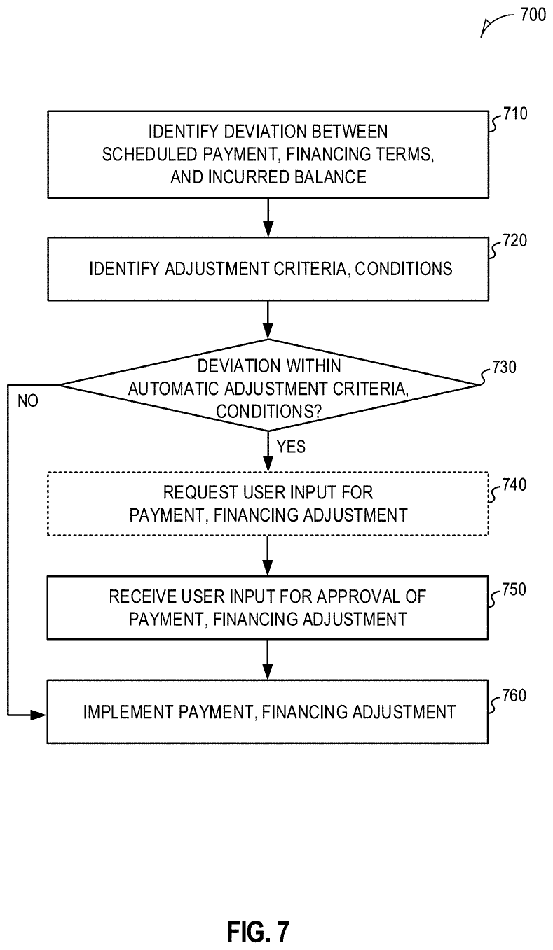

FIG. 7 is a flowchart 700 of an example method of reconciling and managing data for dynamic payment modification. As one example of dynamic payment modification, if a patient balance for a specific visit deviates from the estimate, the data processing system will attempt to dynamically adjust the financing terms established for the visit, using a defined set of boundaries (e.g., boundaries agreed to by patient, defined by the provider, defined by regulations, etc.).

The flowchart 700 begins with the identification of a deviation between a scheduled payment, financing terms, and an incurred balance (operation 710). At some point (e.g., after the healthcare service is performed and entered in a billing system), the financed visit will change from an estimate to an actual patient balance. In some examples, a pre-service estimate (“front-end”) may be connected to a corresponding post-service visit (“back-end”) once the visit has a patient balance associated with it. Thus, once the visit is adjudicated through the payer and becomes a patient obligation, the data processing system can identify that the balance deviates from the estimate.

The flowchart 700 continues with the identification of adjustment criteria and conditions (operation 720). If the post-adjudication balance and estimates deviate (absolutely or by some margin), (decision 730), then the data processing system will automatically adjust the recurring payment amounts and/or durations based on adjustment criteria and conditions (operation 760). For instance, the criteria and conditions include identifying and applying adjustments within state and federal lending compliance laws or regulations.

Certain deviations (e.g., in magnitude or direction) may optionally require the patient to accept new payment terms. The data processing system may coordinate a communication to and provide a mechanism to enable the patient to accept the new terms online before changing them. In some scenarios, the user may provide input for the type of modification (e.g., to select a reduced number of payments, reduced payment amount, etc.); in other scenarios, this modification is automatically selected and implemented for the patient. This is indicated in the flowchart 700 with an optional request for user input for payment or financing adjustment (operation 740) and the receipt of user input for approval of the payment and financing adjustment (operation 750).

FIG. 8 is an illustration of a user interface 800, providing example patient user interface features for implementing payment modification for front-end payment management for a patient account. Similar to the user interface 600B depicted in FIG. 6E, the user interface 800 includes financing plan functionality 802, which presents terms 812 of a financing plan, a list of visits 813 applicable to the financing plan, and balance information 814 applicable to the financing plan.

The user interface 800 also includes adjustment details 811 which indicate a modification to the financing plan. This modification is indicated as being automatically implemented (e.g., reducing the duration of the financing plan, due to a balance decrease for the visit). In other examples, such as where a modification results in an increased balance or payment terms, the user interface 800 may provide a user-selectable control or input feature to allow confirmation of the modification. Other features to select, modify, or reject the modification may also be provided within the user interface 800 or in like screens.

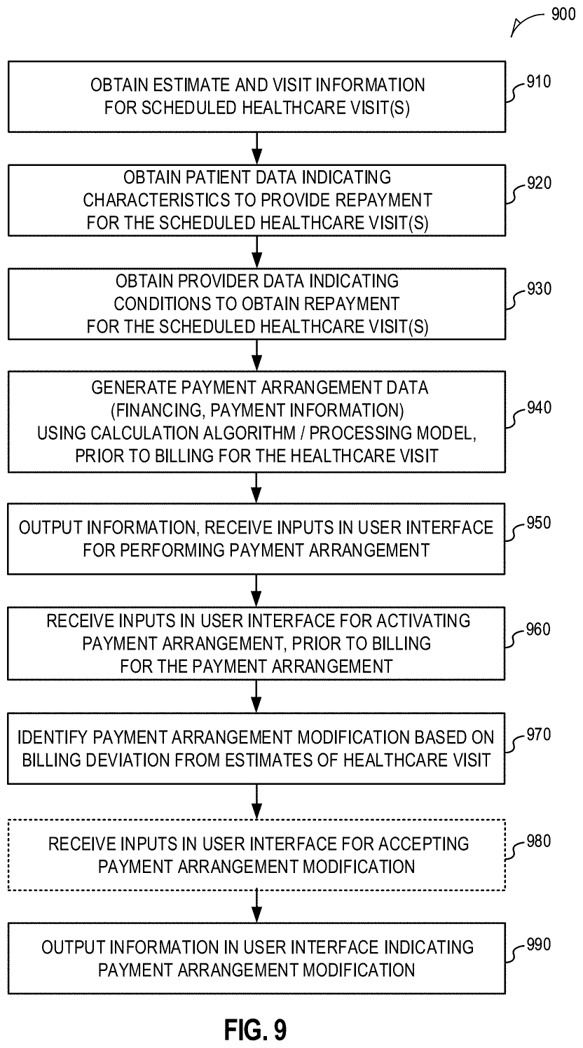

FIG. 9 is a flowchart 900 of an example method of originating and adjusting front-end healthcare payment data representations. The flowchart 900 commences by obtaining estimate and visit information (operation 910), patient data (operation 920) (including financial, credit, and demographic data for the patient), and provider data (operation 930), in connection with a scheduled healthcare visit (or visits). In an example, the estimate data is obtained from an estimate data source regarding an estimated balance of a healthcare visit, for a healthcare visit scheduled to be provided for a patient at a healthcare provider. In an example, the patient data is obtained from a patient information data source, and indicates characteristics of the patient to provide payment for the estimated balance of the scheduled healthcare visit. In an example, the provider data is obtained from a provider information data source, and indicates payment conditions of the provider to obtain payment for the estimated balance of the scheduled healthcare visit, to be transferred to an actual balance after billing for the scheduled healthcare visit.

The flowchart 900 continues by generating payment arrangement data, relating to financing, scheduled payments, and the like, using a calculation algorithm, trained data processing model, or other data analysis module (operation 940). This data is used to establish a payment arrangement prior to the billing for the healthcare visit. For instance, a trained model may be configured to evaluate the characteristics of the patient and the payment conditions of the healthcare provider, such as where the model determines (identifies, provides, classifies, etc.) parameters of the payment arrangement for the payment for the estimated balance of the scheduled healthcare visit. In various examples, the trained model may be a machine learning or neural network model trained on a labeled data set consisting of myriad variables including, but not limited to, consumer demographic data, consumer credit data, behavioral information, and healthcare related information (including but limited to transactional data and visit data), or other data values discussed herein.

The flowchart 900 then proceeds by outputting or providing output information, for the estimated balance and the payment operation, for use in a user interface (operation 950). This output may be accompanied by inputs which receive, specify, update, activate, approve, or schedule the payment arrangement (operation 960). Such outputs and inputs may be provided in a user interface that is rendered or adapted for use by a healthcare provider, a patient/guarantor, an agent or service provider on behalf of either entity, or the like.

The flowchart 900 concludes with operations occurring in the event of a payment modification scenario, where a deviation is identified between the estimated balance and the actual balance, after the billing for the scheduled healthcare visit. This may include, identifying and implementing a payment arrangement modification based on the deviation (operation 970), and outputting information which indicates the payment arrangement modification (operation 990). In some examples, such as where the deviation leads to an increase in payment terms, inputs may optionally be received in the user interface for accepting the modification (operation 980).

In various examples, the outputting of the information (e.g., with operation 990) may be provided as part of providing remote access, to a user interface, to a patient or a patient's guarantor over a network. A variety of data values that indicate the information for the payment arrangement or the payment arrangement modification may be transferred, combined, aggregated, rendered, and displayed via this user interface, including via websites, apps, and in interfaces of health information systems. As is apparent, this outputting of the information, and the creation or provision of a user interface and user interface functionality (e.g., with functionality indicated in FIGS. 6A-6F), provides a new form of interactivity with combined healthcare and financial information that is not available in conventional payment or billing systems.

Other variations to the data processing may involve converting or translating one or more of the obtained data values (e.g., obtained with operations 910, 920, 930) to a standardized or normalized format. For instance, obtaining the patient data, provider data, and estimate data may be obtained from multiple different interfaces with different provider/estimate/patient systems. The obtained data may be converted and consolidated, and temporarily or persistently stored, cached, or persisted for use with the algorithms or processing models discussed herein. Beyond the use of the workflows detailed in FIG. 9, such consolidated information may be communicated or made available to other healthcare data processing entities and systems.

Further variations to the workflows described above may allow integration of the present data processing systems to other variations of front-end payment processing and payment estimates. For example, the workflows may be modified receive deposits from users, over time, to create a pre-funded credit balance that is held by the provider as security in advance of a scheduled procedure. For instance, providers and patients may develop a target aggregate funding amount and then establish a periodic funding plan whereby a payment service provider will automatically and consistently deduct specific amounts from a patient user's funding source (e.g. checking account, credit card account, etc.) according to a specific pre-funding schedule established by the patient user. Until such time as a specific balance amount is pre-funded and provider charges are incurred, the credit balance can be requested for release back to the patient with an automated deposit to the source funding account. However, once the patient authorizes treatment by the relevant provider, the credit balance funds become locked and can only be used to cover patient obligations that will become due on the associated, and duly authorized procedure.

Also in a further variation, the workflows discussed above may include functionality to automatically (or, based on user input or control) cause a lock, guarantee, or preservation of some attribute of a financing plan or estimate. For example, this functionality may allow a lock of the finance plan interest rate based on the estimate price. As an example, if the estimate ends up being higher than expected, which results in a longer plan duration which would have a higher interest rate associated with it, the system may honor the original interest rate that has been obtained with the “lock”.

Also in a further variation, the obligation or agreement for repayment may be expressed as an asset (or derivatives or variations of this asset) that can be brokered, sold, or transferred. For instance, the system may include functionality to enable a third party to purchase asset rights from the provider for an estimated visit, prior to the visit occurring with the patient. In an example, this asset transfer may involve brokering the payment for the asset, which may include a discount for the estimate balance (e.g., discounted relative to debt balance). This may also involve creating and managing an online auction process for the sale or transfer of the asset (or portions of the asset).

Further, intelligent decisioning and logic may be used to affect brokering, sale, or transfer of such assets. For example, a decisioning engine may be used to allow an originating health system to choose whether to sell an estimated visit asset to a third party. This decisioning engine may utilize machine learning and/or predictive analytics to help the health system perform actions with such assets to maximize profitability. The decisioning engine may also advise the system not to sell or transfer certain assets due to non-financial issues, such as based on information identified with a rules engine (e.g., based on identified reputation risk, certain types of procedures that are more likely to lead to bad clinical outcomes or death, or similar considerations).

In some examples, the presently disclosed embodiments access data (e.g., extract, obtain, collect, receive, or otherwise access data), transform the data, and load the data to generate longitudinal portrayals of the data. A longitudinal portrayal may be cohort-based, where a cohort is a group of guarantor accounts (within a billing system), balances, and/or visits that share a common origination characteristic and are tracked longitudinally over time. Accordingly, a number of the data visits discussed herein may be provided in a longitudinal portrayal that includes multiple transactions affecting the balances of a grouping of accounts as tracked over a period of time. The grouping of accounts may include accounts (e.g. one or more accounts) from independent billing systems and/or balance sheet entities.

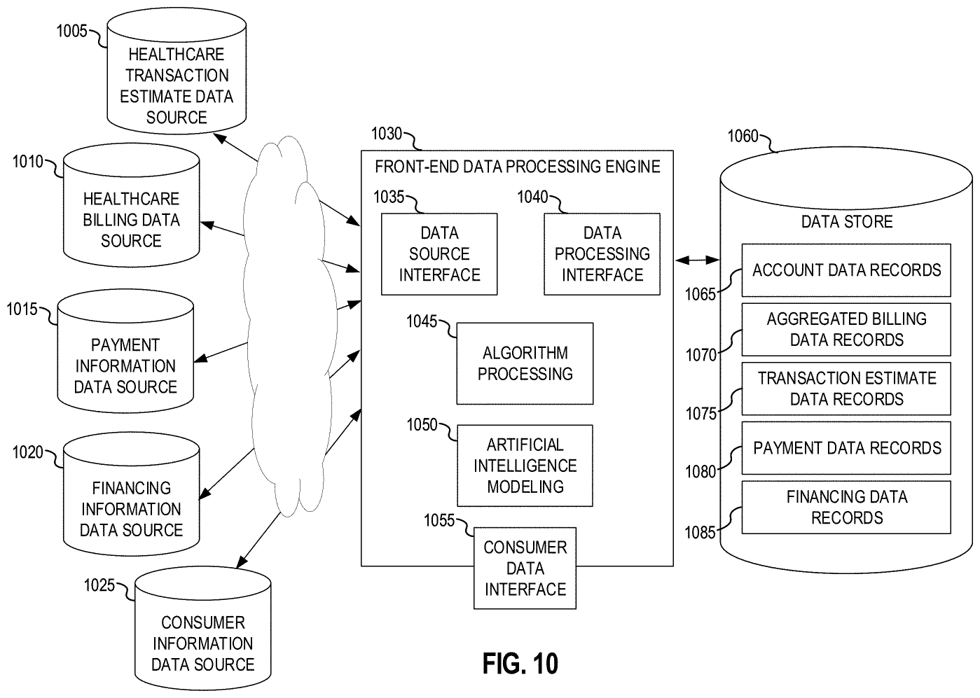

FIG. 10 is a schematic of data sources and data records operating in a data processing system. A front-end data processing engine 1030 is depicted as including: a data source interface 1035 to collect data from among multiple data sources; a data processing interface 1040 to store and access data for processing uses; algorithm processing functionality 1045; artificial intelligence modeling functionality 1050; and a consumer data interface 1052 and provider data interface 1054. For instance, the respective user interfaces discussed above (e.g., for consumers or providers, or similar entities) may communicatively couple with the consumer data interface 1052 and the provider data interface 1054, to obtain data for user inputs and provide commands for processing.

FIG. 10 also provides a high-level overview of the types of data sources that may be involved. These may include, a healthcare transaction estimate data source 1005, a healthcare billing data source 1010, a payment information data source 1015, a financing information data source 1020, and a consumer information data source 1025. Any of these data sources may be provided by the same or other entities, including via network-accessed services.

FIG. 10 finally provides a high-level overview of the types of information which may be stored, maintained, or tracked by the data processing system, within data store 1060. The various types of data records may include, account data records 1065, aggregated billing data records 1070, transaction estimate data records 1075, payment data records 1080, or financing data records 1085. In various examples, these data records may be maintained within a private data store, or within a data store associated with a coupled information system (e.g., billing system, payment management system, etc.)

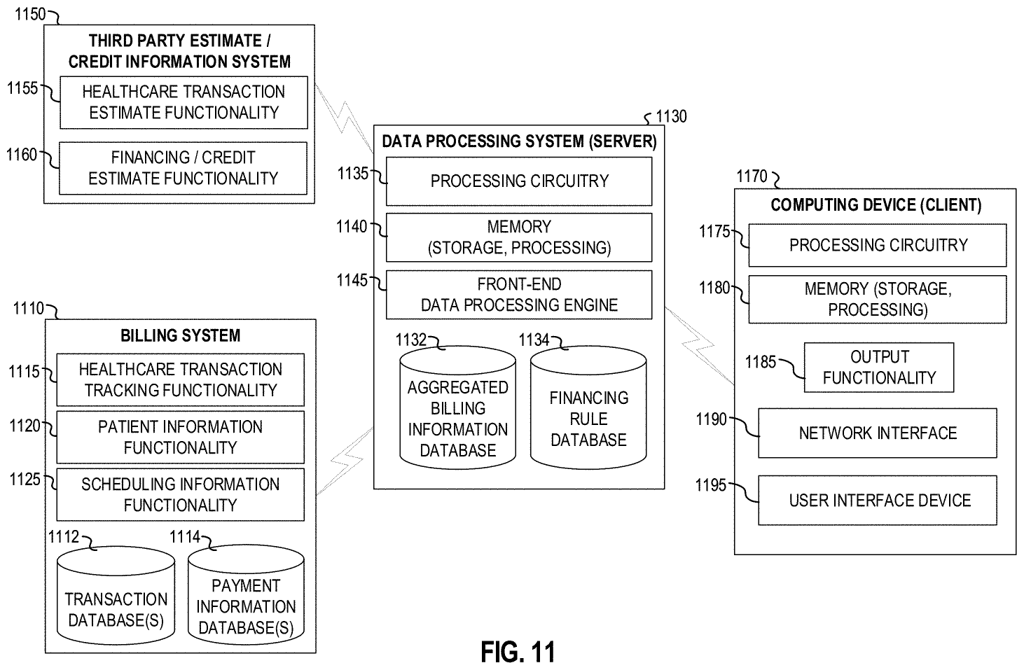

FIG. 11 illustrates a schematic of operational and functional components used among devices and systems to implement the presently disclosed techniques. These components include a data processing system 1130, a computing device 1170, a billing system 1110, and a third party estimate or credit information system 1150, and various sub-components that are included or associated with the front end payment processing functionality discussed herein.

The data processing system 1130 may be an example of a server that implements any of the payment estimate calculation, identification, scheduling, or coordinating services or features discussed herein (including providing features of relevant user interface and workflows for front end financing operations). The computing device 1170 may be an example of a computing platform operating as a client (e.g., by a patient/consumer, or a provider/agent) to present user interfaces, receive inputs, and control the workflows and data operations discussed herein. The billing system 1110 may be an example of a billing information source providing scheduled visit or actual visit information.

The computing systems 1130, 1170 may respectively include processing circuitry 1135, 1175, and memory 1140, 1180 to facilitate execution of instructions of respective processing functions. This may include software instructions associated with a front-end data processing engine 1145, and output functionality 1185. The computing device 1170 is also specifically depicted as including a network interface 1190 and a user interface 1195, although such components may also be present in other systems and subsystems in FIG. 11.

The third party estimate or credit information system 1150 is depicted as including healthcare transaction estimate functionality 1155 and financing or credit estimate functionality 1160. In some examples, aspects of estimate data processing, financing or credit calculations, or the like may be computed at the information system 1150 or another third party with such functionality.

The billing system 1110 is depicted as including healthcare transaction functionality 1115, patient information functionality 1120, and scheduling information functionality 1125. In some examples, aspects of healthcare data collection, compilation, filtering, or identification of certain transactions or scheduled events occurs at the billing system 1110 or another third party with such functionality.

A limited number of databases are shown in FIG. 11, including aggregated billing information database 1132 and financing rule data base 1134 associated with the data processing system 1130; and transaction database 1112, and payment information database 1114 associated with the billing system 1110. Other database or data store formats and systems may also be distributed within FIG. 11.

The components shown in FIG. 11 may be embodied or implemented in hardware, software, or any combination thereof. The functionality of each component is one example arrangement of functionality and one of ordinary skill with the benefit of the present disclosure will appreciate that other organizations are possible. For example, one or more of the functions components depicted in the data processing system 1130 may be distributed among other components or systems. Likewise, one or more of the computing device 1170 components may be distributed into other configurations or among multiple systems.

It will be understood that embodiments discussed herein may include various steps, which may be embodied in machine-executable instructions to be executed by a general-purpose or special-purpose computer (or other electronic device). Alternatively, the steps may be performed by hardware components that include specific logic for performing the steps, or by a combination of hardware, software, and/or firmware.

Embodiments may also be provided as a computer program product including a computer-readable storage medium having stored instructions thereon that may be used to program a computer (or other electronic device) to perform processes described herein. The computer-readable storage medium may include, but is not limited to: hard drives, floppy diskettes, optical disks, CD-ROMs, DVD-ROMs, ROMs, RAMs, EPROMs, EEPROMs, magnetic or optical cards, solid-state memory devices, or other types of media/machine-readable media suitable for storing electronic instructions.

As used herein, a software module or component may include any type of computer instruction or computer-executable code located within a memory device and/or computer-readable storage medium. A software module may, for instance, comprise one or more physical or logical blocks of computer instructions, which may be organized as a routine, a program, an object, a component, a data structure, etc., that perform one or more tasks or implement particular abstract data types.

In certain embodiments, a particular software module may comprise disparate instructions stored in different locations of a memory device, which together implement the described functionality of the module. Indeed, a module may comprise a single instruction or many instructions, and may be distributed over several different code segments, among different programs, and across several memory devices. Some embodiments may be practiced in a distributed computing environment where tasks are performed by a remote processing device linked through a communications network. In a distributed computing environment, software modules may be located in local and/or remote memory storage devices. In addition, data being tied or rendered together in a database record may be resident in the same memory device, or across several memory devices, and may be linked together in fields of a record in a database across a network.

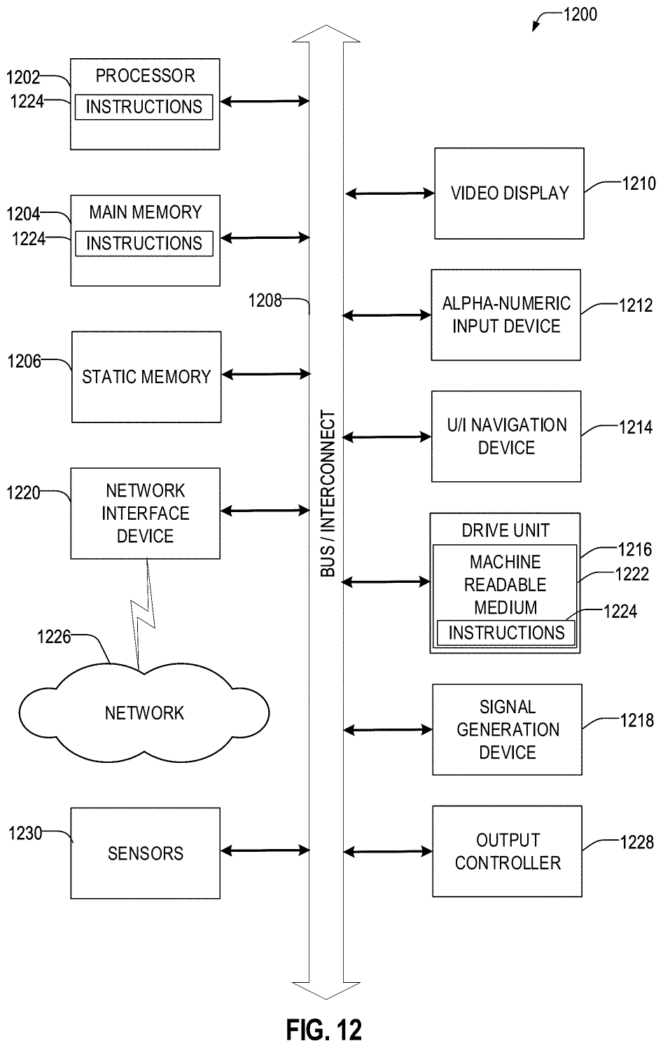

FIG. 12 illustrates a block diagram of an example machine 1200 upon which any one or more of the techniques (e.g., methodologies) discussed herein may perform. In alternative embodiments, the machine 1200 may operate as a standalone device or may be connected (e.g., networked) to other machines. In a networked deployment, the machine 1200 may operate in the capacity of a server machine, a client machine, or both in server-client network environments. In an example, the machine 1200 may act as a peer machine in peer-to-peer (P2P) (or other distributed) network environment. The machine 1200 may be a computing device such as a personal computer (PC), a tablet PC, a personal digital assistant (PDA), a mobile telephone, a cloud-based or networked server, a virtualized server, a smart phone, a web appliance, a network router, an access point, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations. Machine 1200 or a variant thereof may implement the devices and platforms 130, 135, 140, 1030, 1110, 1130, 1150, 1170, the operations provided by the methods of FIGS. 3, 5, 7, and 9, or features of any of the user interfaces described or depicted herein.

Examples, as described herein, may include, or may operate on, logic or a number of components, modules, or mechanisms (hereinafter “components”). Such components are tangible entities (e.g., hardware) capable of performing specified operations and may be configured or arranged in a certain manner. In an example, circuits or circuitry may be arranged (e.g., internally or with respect to external entities such as other circuits or circuitry) in a specified manner as a component. In an example, the whole or part of one or more computer systems (e.g., a standalone, client or server computer system) or one or more hardware processors may be configured by firmware or software (e.g., instructions, an application portion, or an application) as a component that operates to perform specified operations. In an example, the software may reside on a machine readable medium, such as a non-transitory machine-readable storage medium. In an example, the software, when executed by the underlying hardware of the component, causes the hardware to perform the specified operations.

Accordingly, such a component encompasses a tangible entity, be that an entity that is physically constructed, specifically configured (e.g., hardwired), or temporarily (e.g., transitorily) configured (e.g., programmed) to operate in a specified manner or to perform part or all of any operation described herein. Considering examples in which components are temporarily configured, each of the components need not be instantiated at any one moment in time. For example, where the components comprise a general-purpose hardware processor configured using software, the general-purpose hardware processor may be configured as respective different components at different times. Software may accordingly configure a hardware processor, for example, to constitute a particular component at one instance of time and to constitute a different component at a different instance of time.

Machine (e.g., computer system) 1200 may include a hardware processor 1202 (e.g., a central processing unit (CPU), a graphics processing unit (GPU), a hardware processor core, or any combination thereof), a main memory 1204 and a static memory 1206, some or all of which may communicate with each other via an interlink (e.g., bus) 1208. The machine 1200 may further include a display unit 1210, an alphanumeric input device 1212 (e.g., a keyboard), and a user interface (UI) navigation device 1214 (e.g., a mouse). In an example, the display unit 1210, input device 1212 and UI navigation device 1214 may be a touch screen display. The machine 1200 may additionally include a storage device (e.g., drive unit) 1216, a signal generation device 1218 (e.g., a speaker), a network interface device 1220, and one or more sensors 1230, such as a global positioning system (GPS) sensor, compass, accelerometer, or other sensor. The machine 1200 may include an output controller 1228, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

The storage device 1216 may include a machine readable medium 1222 on which is stored one or more sets of data structures or instructions 1224 (e.g., software) embodying or utilized by any one or more of the techniques or functions described herein. The instructions 1224 may also reside, completely or at least partially, within the main memory 1204, within static memory 1206, or within the hardware processor 1202 during execution thereof by the machine 1200. In an example, one or any combination of the hardware processor 1202, the main memory 1204, the static memory 1206, or the storage device 1216 may constitute machine readable media.

While the machine readable medium 1222 is illustrated as a single medium, a machine-readable medium may include a single medium or multiple media (e.g., a centralized or distributed database, and/or associated caches and servers) configured to store the one or more instructions 1224. Thus, the term machine readable medium may include any medium that is capable of storing, encoding, or carrying instructions for execution by the machine 1200 and that cause the machine 1200 to perform any one or more of the techniques of the present disclosure, or that is capable of storing, encoding or carrying data structures used by or associated with such instructions.

Non-limiting machine readable medium examples may include solid-state memories, and optical and magnetic media. Specific examples of machine readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; Random Access Memory (RAM); Solid State Drives (SSD); and CD-ROM and DVD-ROM disks. In some examples, machine readable media may include non-transitory machine-readable media (e.g., excluding a transitory propagating signal).

The instructions 1224 may further be transmitted or received over a communications network 1226 using a transmission medium via the network interface device 1220. The machine 1200 may communicate with one or more other machines utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks, such as networks implementing the Institute of Electrical and Electronics Engineers (IEEE) 802.11 family of standards known as Wi-Fi®, a Long Term Evolution (LTE) family of standards, a Universal Mobile Telecommunications System (UMTS) family of standards, peer-to-peer (P2P) networks, among others. In an example, the network interface device 1220 may include one or more physical jacks (e.g., Ethernet, coaxial, or phone jacks) or one or more antennas to connect to the communications network 1226. In an example, the network interface device 1220 may include a plurality of antennas to wirelessly communicate using at least one of single-input multiple-output (SIMO), multiple-input multiple-output (MIMO), or multiple-input single-output (MISO) techniques. In some examples, the network interface device 1220 may wirelessly communicate using Multiple User MIMO techniques.

The above description provides numerous specific details for a thorough understanding of the embodiments described herein. However, those of skill in the art will recognize that one or more of the specific details may be omitted, or other methods, components, or materials may be used. In some cases, operations are not shown or described in detail.

Furthermore, the described features, operations, or characteristics may be combined in any suitable manner in one or more embodiments. It will also be readily understood that the order of the steps or actions of the methods described in connection with the embodiments disclosed may be changed as would be apparent to those skilled in the art. Thus, any order in the drawings or Detailed Description is for illustrative purposes only and is not meant to imply a required order, unless specified to require an order.

What is claimed is:

1. A computing system, comprising:
memory circuitry;
processor circuitry; and
a storage medium including instructions that, when executed by the processor circuitry and the memory circuitry of the computer system, implement data processing operations, comprising:
obtaining, from an estimate data source, estimate data relating to an estimated balance of a healthcare visit, the healthcare visit scheduled to be provided for a patient at a healthcare provider;
obtaining, from a patient information data source, patient data indicating characteristics of the patient or a guarantor of the patient to provide payment for the estimated balance of the scheduled healthcare visit;
obtaining, from a provider information data source, provider data indicating payment conditions of the provider to obtain payment for the estimated balance of the scheduled healthcare visit, to be transferred to an actual balance after billing for the scheduled healthcare visit;
generating, using a machine learning data processing model, data to establish a payment arrangement prior to the billing for the healthcare visit, the model trained to evaluate the characteristics of the patient and the payment conditions of the healthcare provider, wherein the model outputs parameters of the payment arrangement for the payment for the estimated balance of the scheduled healthcare visit; and
providing information, for the estimated balance and the payment arrangement, for output in a user interface.

2. The computing system of claim 1, the data processing operations further comprising:
rendering the user interface, the user interface including user input controls to receive patient input to specify at least one of the parameters of the payment arrangement, and to schedule the payment arrangement.

3. The computing system of claim 2, the data processing operations further comprising:
detecting a deviation between the estimated balance and the actual balance, after the billing for the scheduled healthcare visit;
identifying a modification to the payment arrangement, using the machine learning data processing model, the model configured to generate updated parameters for the payment for the actual balance of the scheduled healthcare visit; and
implementing the modification to the payment arrangement, to utilize the updated parameters for subsequent payment of the actual balance.

4. The computing system of claim 3, wherein the deviation occurs within a predefined range, and wherein implementing the modification to the payment arrangement includes automatically updating at least one payment scheduled of the payment arrangement.

5. The computing system of claim 3, the data processing operations further comprising:
rendering information, in the user interface, indicating the modification to the payment arrangement, the user interface including user input controls to receive input from the patient or the guarantor of the patient to accept the modification to the payment arrangement.

6. The computing system of claim 1, wherein the estimate data, patient data, and provider data are obtained from the respective data sources via a network, and wherein the user interface is accessible to the patient or the guarantor of the patient.

7. A method of healthcare data processing to generate information for a user-interactive graphical user interface, comprising electronic operations implemented with processor circuitry of a computing system, and the electronic operations comprising:
obtaining, from an estimate data source, estimate data relating to an estimated balance of a healthcare visit, the healthcare visit scheduled to be provided for a patient at a healthcare provider;
obtaining, from a patient information data source, patient data indicating characteristics of the patient or a guarantor of the patient to provide payment for the estimated balance of the scheduled healthcare visit;
obtaining, from a provider information data source, provider data indicating payment conditions of the provider to obtain payment for the estimated balance of the scheduled healthcare visit, to be transferred to an actual balance after billing for the scheduled healthcare visit;
generating, using a machine learning data processing model, data to establish a payment arrangement prior to the billing for the healthcare visit, the model trained to evaluate the characteristics of the patient and the payment conditions of the healthcare provider, wherein the model outputs parameters of the payment arrangement for the payment for the estimated balance of the scheduled healthcare visit; and
providing information, for the estimated balance and the payment arrangement, for output in a user interface.

8. The method of claim 7, the electronic operations further comprising:
rendering the user interface, the user interface including user input controls to receive patient input to specify at least one of the parameters of the payment arrangement, and to schedule the payment arrangement.

9. The method of claim 8, the electronic operations further comprising:
detecting a deviation between the estimated balance and the actual balance, after the billing for the scheduled healthcare visit;
identifying a modification to the payment arrangement, using the machine learning data processing model, the model configured to generate updated parameters for the payment for the actual balance of the scheduled healthcare visit; and
implementing the modification to the payment arrangement, to utilize the updated parameters for subsequent payment of the actual balance.

10. The method of claim 9, wherein the deviation occurs within a predefined range, and wherein implementing the modification to the payment arrangement includes automatically updating at least one payment scheduled of the payment arrangement.

11. The method of claim 9, the electronic operations further comprising:
rendering information, in the user interface, indicating the modification to the payment arrangement, the user interface including user input controls to receive input from the patient or guarantor of the patient to accept the modification to the payment arrangement.

12. The method of claim 7, further comprising:
providing remote access to the user interface, wherein the user interface is accessible to the patient or the guarantor of the patient.

13. The method of claim 7, wherein the user interface is accessible to an agent of the healthcare provider.

14. The method of claim 7, wherein the estimate data source, the patient information data source, and the provider information data source are accessed via a network, using respective application programming interfaces, wherein the estimate data source is provided by a third party financial estimate system, wherein the patient information data source is provided by a medical information system, and wherein the provider information data source is provided by a billing system associated with the healthcare provider.

15. A non-transitory computer-readable storage medium having stored thereon instructions that, when executed by a computing system, cause the computing system to provide a user-interactive graphical user interface with operations comprising:
obtaining, from an estimate data source, estimate data relating to an estimated balance of a healthcare visit, the healthcare visit scheduled to be provided for a patient at a healthcare provider;
obtaining, from a patient information data source, patient data indicating characteristics of the patient or a guarantor of the patient to provide payment for the estimated balance of the scheduled healthcare visit;
obtaining, from a provider information data source, provider data indicating payment conditions of the provider to obtain payment for the estimated balance of the scheduled healthcare visit, to be transferred to an actual balance after billing for the scheduled healthcare visit;
generating, using a machine learning data processing model, data to establish a payment arrangement prior to the billing for the healthcare visit, the model trained to evaluate the characteristics of the patient and the payment conditions of the healthcare provider, wherein the model outputs parameters of the payment arrangement for the payment for the estimated balance of the scheduled healthcare visit; and
outputting, in the graphical user interface, information for the estimated balance and the payment arrangement.

16. The computer-readable storage medium of claim 15, the operations further comprising:
rendering the user interface, the user interface including user input controls to receive patient input to specify at least one of the parameters of the payment arrangement, and to schedule the payment arrangement.

17. The computer-readable storage medium of claim 16, the operations further comprising:
detecting a deviation between the estimated balance and the actual balance, after the billing for the scheduled healthcare visit;
identifying a modification to the payment arrangement, using the machine learning data processing model, the model configured to generate updated parameters for the payment for the actual balance of the scheduled healthcare visit; and
implementing the modification to the payment arrangement, to utilize the updated parameters for subsequent payment of the actual balance.

18. The computer-readable storage medium of claim 17, wherein the deviation occurs within a predefined range, and wherein implementing the modification to the payment arrangement includes automatically updating at least one payment scheduled of the payment arrangement.

19. The computer-readable storage medium of claim 17, the operations further comprising:
rendering information, in the user interface, indicating the modification to the payment arrangement, the user interface including user input controls to receive input from the patient or the guarantor of the patient to accept the modification to the payment arrangement.

20. The computer-readable storage medium of claim 15, wherein the user interface is accessible to the patient or the guarantor of the patient.

21. The computer-readable storage medium of claim 15, wherein the user interface is accessible to an agent of the healthcare provider.

22. The computer-readable storage medium of claim 15, wherein the estimate data source, the patient information data source, and the provider information data source are accessed via a network, wherein the estimate data source is provided by a third party financial estimate system, wherein the patient information data source is provided by a medical information system, and wherein the provider information data source is provided by a billing system associated with the healthcare provider.
